SEQUENCE LISTING (16 sequences)

SEQ ID NO: 1 - protein, 1360 aa, Artificial Sequence
  It is synthesized.
TKLGKPYGIGVDIGSNSIGFAAVDENSHLIRLKGKTVIGARLFEEGKAAADRRASRTTRRRLSRNRWRLSFLRDFFESHITPTGPNFFMRQKYSEISPKDKARYKYEKRLFNDRTDAEFYQQYPTMYHLRNRLLTDPSKADVREIYFAIHHILKSRGHFLTSGYAKDFNTNKVALNEIFPALQDAYAQVYPDLDITFDENKVNEFKTVLLNEKATPSDTQRALVNLLLAEDGDKDILKQQKQVLTEFAKAVVGLKTKLNVALGTEVDSSEATVWNFSLGQLDDKWAGIESAMTDEGTEILDQIRDLYRARLLNGIVPAGKTLSQAKVDDYAQHHEDLELFKDYLKQLEDDGTAKAIRQLYDRYIDGDDAKPFVREDFVKALTKEVTAHPNAKSPELLERLAQPDFMLKQRNKANGAIPVQMQQRELDQIIKNQSVYYDWLAAPNPVEKHRKSMPYQLDELLNFRIPYYVGPLVTAKEQKAAQGGVFAWMVRKDPDGNITPYNFDEKVDREASANTFIQRMKTTDTYLIGEDVLPKQSLLYQRYEVLNELNNVRVNNEKLSIEQKQQVIRELFERHNSVTIKQFAENLRAHGDYAHIPEIRGLADEKRFLSSLSTYRQLKSLLPDAIDDPAKQADIENIIAWSTVFEDAAIFKTKLKEINWLDSQTITKLSNIRYRGWGQFSHKFLNGLTLGNGHTIIQELFLSNNNLMQILTDETLQKKMTELNADKLKAANINDAIDDAYTSPSNKKALRQVLRVIEDIKRAADGQDPSWLYVETADGGGTPGKRTRARQHQLQEIYANAAHELIDSAVRGELEDKIADKADFNDRLVLYFMQGGRDIYTGTPLNIDQLSSYDIDHILPQSLIKDNSLDNRVLVHAIINREKNATFASTIYAQKMNATWRQWHEAGLISGRKLRNLQMRPDQIDKYASGFVARQLVETRQIIKLTEQIVAAQYPETKIIAVKAGLSHQLREELEFPKNRDVNHYHHAFDAFLAARIGTYLLKRFPNLQAFFTYGKFKKADVKKLRGFNFIRDITHAEDKIVVKDTGEVIWDKKRDVDELDRIYNFKRMLITHEVHFEKADLFKQTVYGAKDSKEAGGKKQLISKKKGYPVDIYGGYTQETGSYLSVVRLTKKAMYAVVKVSTRDAAKLAVAKSISEQKENETLNDIIDEKLSKISKNGKITHELFEVVLPRVGQKTLFKNSKYNLFLVNSDTYMHNYQELWMPREYQKMWKDISLSKYGDAQTEDQLDQIFGFIVSQVNSYFNLYDINQFRKKLNDAADKFATLPMRDTDDMQGKIATIGQLLIGLQANAARSDLRNLDIKTPLGLLQIPNGISLDSDTSVVYQSPTGLLEREVHLSDL

SEQ ID NO: 2 - RNA, 102 nt, Artificial Sequence
  It is synthesized.
  misc_feature (1)..(20): n is a, c, g, or u
nnnnnnnnnnnnnnnnnnnngucucagguagcgaacuacacguugagaucaaacaaagcuucggcugaguuucaauuuuugagcccauguugggccauacau

SEQ ID NO: 3 - DNA, 4086 bp, Lactobacillus paracasei
atgacaaagttaggtaaaccatatggcattggagtcgatatcggctcaaactcgattggatttgcggctgtggatgagaacagtcatcttattcgattgaagggaaaaacggttattggggcaagactgtttgaagaaggaaaagcagcagcggatcgccgagcaagccgtaccactcgccgtcgtttgagtcgaaatcgttggcggctcagttttttgcgtgatttttttgaatcgcatatcacgcctactggcccgaatttcttcatgcgtcaaaagtactcggaaatctcgccgaaggacaaggcccgttacaagtatgagaagcggctttttaatgatcggacggatgctgagttttaccaacagtatccaacgatgtaccatttacgcaatcggcttttgacagatccaagcaaagccgatgttcgcgagatttattttgccattcatcatattttgaaaagtcgcgggcactttttgacgtctgggtatgccaaagattttaatacaaacaaggtcgcgttaaacgagattttccctgcattgcaggatgcctacgctcaagtttatccggatctggacatcacttttgatgagaacaaggtgaatgagtttaaaaccgttctgttgaatgagaaagcgacgccaagtgacacgcaacgagctttagtcaatcttttgttggctgaagatggcgacaaggatatattgaagcagcagaagcaggttttaactgagtttgctaaagcagtggtcggactgaaaacgaagctcaatgtggcgctagggaccgaagttgatagttctgaggccactgtttggaacttttcattagggcagcttgacgataaatgggcaggcattgagtcagccatgactgatgaaggtaccgaaattctggaccagattcgtgatctttatcgagctcggctccttaatggtattgtgcccgcaggaaagacgctttctcaggcaaaggttgatgattatgcacaacatcatgaagacctcgaattgttcaaagactatttaaaacaacttgaagatgatggaacggctaaagcaatccgacagctttacgatcgctatatagacggtgacgatgctaagccatttgtacgtgaagattttgttaaagcgttaactaaagaagtgacggctcatccaaacgccaagtctcctgaactgcttgaacgtttggcgcaaccggacttcatgcttaagcaacgcaacaaagcaaacggggctattccagtccaaatgcaacagcgggaattggatcagatcatcaaaaatcagtctgtttattatgactggctggctgcgccaaatccggtggaaaagcatcggaaaagtatgccttatcagttggatgaattgcttaattttcgaattccgtattacgttggaccattggtgacggctaaagagcaaaaggcagcacaaggcggggtttttgcttggatggttcggaaagatcctgatggtaatatcacgccttataattttgacgaaaaggtagatcgcgaagcttcggctaatacctttattcagcggatgaagacgaccgacacatacttgattggtgaagacgtgttgcctaagcaaagtttgctgtatcaacgctatgaggtgttgaatgaactgaataatgttcgtgttaataacgagaagcttagcatcgaacaaaaacaacaagttattcgagaactgtttgaacggcataatagcgtgacaattaaacaatttgccgaaaatctgcgagcgcatggtgattacgcccacattcctgaaattcgtggtttagctgatgagaaacgttttttaagttcactttctacgtaccgtcaactgaaaagtcttttacccgatgcaattgatgatccagccaaacaagccgatattgaaaatatcattgcgtggtcgaccgtttttgaagacgccgcgatttttaaaacgaaattgaaagagatcaactggcttgacagccaaaccattactaagcttagtaacattcgatatcgaggatggggtcagttttcgcataagtttttaaatggcctgacccttggcaacggacataccattattcaagaattgttcctatccaacaacaacttgatgcagatcttaacagacgaaacgctgcaaaagaaaatgaccgagttgaacgccgacaaattgaaagcggccaatatcaatgatgccattgatgatgcgtatacatcaccaagtaataaaaaggcattgcgtcaggtccttcgggttattgaggatattaaacgtgctgctgatggtcaagatcctagttggttgtacgttgagactgcggacggcggtggcacaccaggtaaacgtacacgagcacgccaacatcagcttcaagaaatatatgccaatgcggctcatgaactgattgatagtgcggttcgtggcgaattggaagataaaatcgcagacaaggctgatttcaatgatcgactagtgctgtatttcatgcaaggagggcgtgatatctatactggcacaccactgaatatcgatcaattaagtagttatgatatcgaccatattctgccacagtcactgatcaaagataattctttggataatcgtgtgttagtgcatgccatcatcaaccgagaaaaaaacgcaacatttgcatcaaccatctatgcacaaaagatgaacgcgacatggcgtcaatggcatgaggcagggctgattagcggtcgcaaactgcgtaatctgcaaatgcgtccagatcaaatcgataagtacgcgagcggttttgttgcccgacaattggtggaaactcgacaaattattaaactgacggaacaaattgtagcagcgcagtatcctgaaaccaaaattatcgctgttaaagcaggactttcacatcaattgcgggaagaacttgaatttcctaagaatcgtgatgtgaatcactaccaccatgctttcgatgccttcctagccgcccggattggcacttatttactcaaacgatttcctaatttgcaggctttcttcacgtatggcaagtttaaaaaagccgatgtcaaaaagcttcgcgggttcaactttatccgcgatatcacccatgctgaagataaaattgtggtaaaggataccggcgaagttatttgggataagaagcgggatgttgacgaactggatcggatttacaacttcaagcggatgctgattacgcatgaggtgcactttgagaaggcggatttgtttaagcagacggtttatggggccaaagattcgaaagaggctggagggaagaaacagctcatttcgaagaaaaaaggatatccagttgatatttatggaggttatacgcaagaaacgggtagttatctatctgttgttcgactaacaaaaaaagcgatgtatgctgtcgtgaaagtatcgacgagagatgctgctaaattggcagttgctaagagtatttctgagcaaaaggaaaacgagaccttaaatgatattattgatgaaaaactttcaaagataagcaaaaacggaaaaatcacgcatgagctattcgaggttgtcttaccacgggtggggcaaaaaaccttatttaaaaatagcaaatataatctatttctagtcaactcggatacctacatgcacaattaccaagaactgtggatgccacgtgagtatcagaaaatgtggaaggatatttctctttctaaatacggcgatgcccagacagaagaccagctagatcaaattttcgggtttattgtttctcaagtaaacagttactttaatctgtatgatattaatcaatttcgtaaaaagttgaatgatgcggctgataagtttgctacacttccaatgcgcgatacagatgatatgcaaggaaaaattgcgacaataggtcaactgttaattggccttcaagcaaatgctgcgagaagtgatttaaggaatctcgatataaaaacacctctaggattattgcaaattccaaacggaataagcctagactctgacacttcagttgtatatcaatccccaacaggtttgttggaacgagaagttcatttatctgatctgtaa

SEQ ID NO: 4 - DNA, 4080 bp, Artificial Sequence
  It is synthesized.
accaaactgggcaagccctacggaattggagtggacatcggcagcaacagcatcggatttgctgccgtggacgaaaacagccacctgatcagactgaagggcaagacagtgatcggcgccagactgttcgaagagggaaaggctgccgctgacagaagagctagcagaaccaccagaagaagactgagcagaaacagatggagactgagcttcctgagagacttcttcgagagccacattacacctaccggccccaacttcttcatgagacagaagtacagcgagatcagccccaaggacaaggccagatacaagtacgagaagagactgttcaacgacagaaccgacgccgagttctatcagcagtaccccaccatgtaccacctgagaaacagactgctgaccgatcccagcaaggctgacgtgagagagatctacttcgccatccaccacatcctgaagagcagaggccacttcctgacaagcggatacgccaaggacttcaacaccaacaaggtggccctgaacgagattttccccgctctgcaagatgcttacgcccaggtgtatcccgatctggacatcaccttcgacgagaacaaggtgaacgagttcaagaccgtgctgctgaacgaaaaagccacccccagcgatacacagagagctctggtgaacctgctgctggctgaggatggagacaaggacatcctgaagcagcagaagcaggtgctgaccgagtttgccaaagctgtggtgggcctgaagaccaaactgaacgtggccctgggaaccgaagtggatagcagcgaggctacagtgtggaacttcagcctgggacagctggacgataagtgggctggcatcgaaagcgctatgaccgacgagggaaccgagattctggaccagatcagggacctgtacagagccagactgctgaacggaattgtgcccgctggcaagacactgagccaagccaaggtggacgattacgcccaacaccacgaagacctggagctgttcaaggactacctgaagcagctggaagacgacggaaccgctaaggctatcagacagctgtacgacagatacatcgacggcgacgacgctaagcctttcgtgagagaggacttcgtgaaggccctgaccaaagaggtgaccgctcatcctaacgccaagagccctgagctgctggaaagattagcccagcccgacttcatgctgaagcagagaaacaaggccaacggagccattcctgtgcagatgcagcagagagagctggaccagatcatcaagaaccagagcgtgtactacgactggctggctgctcctaaccctgtggaaaagcacagaaagagcatgccctaccagcttgacgagctgctgaacttcagaatcccctactacgtgggacctctggtgacagccaaggaacagaaggccgctcaaggaggcgtgttcgcttggatggtgagaaaagaccccgacggcaacattaccccctacaacttcgacgagaaggtggacagagaggctagcgccaacaccttcatccagagaatgaagaccaccgacacctacctgatcggcgaagacgtgctgcctaagcagagcctgctgtaccagagatacgaggtgctgaacgagctgaacaacgtgagagtgaacaacgagaagctgagcatcgagcagaagcagcaggtgatcagagagctgttcgagagacacaacagcgtgaccatcaagcagttcgccgagaacctgagagctcacggcgactatgctcatatccccgagattagaggcctggccgacgagaaaagattcctgagcagcctgagcacctacagacagctgaagagcctgctgcctgacgctattgatgatcccgccaagcaagctgacatcgagaacatcatcgcctggagcaccgtgtttgaagacgccgccatcttcaagaccaagctgaaggagatcaactggctggacagccagaccatcaccaagctgagcaacatcagatacagaggctggggccagttctctcacaagttcctgaacggcctgacactgggaaacggccacacaatcatccaggagctgttcctgagcaacaacaacctgatgcagatcctgaccgacgagacactccagaagaagatgaccgagctgaacgccgataaactgaaggccgccaacatcaacgacgccatcgacgacgcttatacaagccccagcaacaagaaggccctgagacaggtgctgagagtgatcgaggacatcaagagagccgccgatggccaagatcctagctggctgtatgtggagacagctgatggaggaggaacacccggaaagagaaccagagccagacagcatcagctgcaggagatctatgctaacgccgcccatgagctgattgatagcgccgtgagaggcgaactggaggacaaaatcgccgacaaggccgacttcaacgacagactggtgctgtacttcatgcagggcggcagagatatctataccggcacccccctgaacattgatcagctgagcagctacgacatcgaccacatccttccccagagcctgatcaaggacaacagcctggacaacagagttctggtgcacgccatcatcaacagagagaagaacgccaccttcgccagcacaatctacgcccagaagatgaacgccacctggagacagtggcatgaggctggactgatcagcggcagaaagctgagaaacctgcagatgaggcccgaccagatcgacaaatacgccagcggattcgtggctagacaactggtggagacaagacagatcatcaagctgaccgagcagattgtggccgctcagtatcccgaaaccaagatcatcgccgtgaaagccggacttagccaccaactgagagaggaactggagttccccaagaacagggacgtgaaccactaccaccacgcctttgatgcttttctggccgccagaattggaacctacctgctgaagagattccccaacctgcaggcctttttcacctacggcaagttcaagaaggccgacgtgaagaagctgagaggcttcaacttcatcagagacatcacccacgccgaggacaagatcgtggtgaaggacaccggcgaagtgatctgggacaagaagagagatgtggacgagctggacagaatctacaacttcaagagaatgctgatcacccacgaggtgcactttgagaaggccgacctgttcaagcaaaccgtgtacggcgctaaggacagcaaagaggctggcggcaagaagcaactgatcagcaagaagaagggctaccccgtggacatctatggaggctacacccaggaaaccggcagctatctgagcgtggtgagactgaccaagaaggccatgtacgccgtggtgaaggtgtctacaagggacgccgctaaactggctgtggccaagagcattagcgagcagaaggagaacgagacactgaacgacatcatcgacgagaagctgagcaagatcagcaagaacggcaagatcacccacgagctgttcgaagtggtgctgcctagagtgggacagaagaccctgttcaagaacagcaagtacaacctgttcctggtgaacagcgacacctacatgcacaactaccaggagctgtggatgcccagagagtaccagaagatgtggaaggacatcagcctgagcaagtacggagatgcccagaccgaggatcaactggaccagatcttcggcttcatcgtgagccaggtgaacagctacttcaacctgtacgacatcaaccagttcagaaagaagctgaacgacgccgctgacaaatttgccaccctgcccatgagagacaccgatgacatgcagggcaagatcgctaccattggccagctgctgatcggactgcaggctaatgctgccagaagcgacctgagaaacctggacatcaagacccctctgggcctgctgcaaatccctaacggcattagcctggacagcgacacaagcgtggtgtaccagagccctacaggactgctggagagagaagtgcacctgagcgacctg

SEQ ID NO: 5 - DNA, 21 bp, Artificial Sequence
  It is synthesized.
gaataacttcgtatagcatac

SEQ ID NO: 6 - DNA, 20 bp, Artificial Sequence
  It is synthesized.
gggaaacgcctggtatcttt

SEQ ID NO: 7 - DNA, 18 bp, Artificial Sequence
  It is synthesized.
cgctccgaaagtttcctt

SEQ ID NO: 8 - DNA, 20 bp, Artificial Sequence
  It is synthesized.
tgctgaagcagagaaacaag

SEQ ID NO: 9 - DNA, 17 bp, Artificial Sequence
  It is synthesized.
tggagacagtggcatga

SEQ ID NO: 10 - DNA, 19 bp, Artificial Sequence
  It is synthesized.
ggtagggcatgctctttct

SEQ ID NO: 11 - DNA, 17 bp, Artificial Sequence
  It is synthesized.
aatctgctcggtcagct

SEQ ID NO: 12 - DNA, 20 bp, Artificial Sequence
  It is synthesized.
caaacaacagatggctggca

SEQ ID NO: 13 - DNA, 21 bp, Artificial Sequence
  It is synthesized.
gatgaacttcagggtcagctt

SEQ ID NO: 14 - DNA, 22 bp, Artificial Sequence
  It is synthesized.
gggacttcctttgtcccaaatc

SEQ ID NO: 15 - DNA, 20 bp, Artificial Sequence
  It is synthesized.
ctcccttttccctcctggca

SEQ ID NO: 16 - DNA, 20 bp, Artificial Sequence
  It is synthesized.
agccagttttggggaggcag

(19) United States
(12) Patent Application Publication
(10) Pub. No.: US 2021/0388392 A1
(43) Pub. Date: Dec. 16, 2021

(54) CRISPR-LpCas9 GENE EDITING SYSTEM AND APPLICATION THEREOF

(71) Applicant: Institute of Food Science and Technology, Chinese Academy of Agricultural Sciences, Beijing (CN)

(72) Inventors: Xiaoyang PANG, Beijing (CN); Weixun LI, Zhijiang (CN); Lan YANG, Changchun (CN); Jiaping LV, Beijing (CN); Jing LU, Beijing (CN); Shuwen ZHANG, Beijing (CN); Qing ZHU, Laohekou (CN); Chen XU, Yantai (CN)

(21) Appl. No.: 17/243,079

(22) Filed: Apr. 28, 2021

(30) Foreign Application Priority Data
Jun. 11, 2020 (CN) 202010531552.0

(51) Int. Cl. C12N 15/90; C12N 9/22; C12N 15/11; C12Q 1/683

(52) U.S. Cl. CPC C12N 15/907; C12N 9/22; C12N 15/11; C12N 2800/22; C12Q 1/683; C12N 2310/20; C12N 2800/80; C12N 15/902

(57) ABSTRACT

The present invention discloses a CRISPR/LpCas9 gene editing system and application thereof, the CRISPR/LpCas9 gene editing system includes a complex of LpCas9 protein and sgRNA, which can accurately locate a target DNA sequence and cleave DNA double strands. The LpCas9 protein has an amino acid sequence shown in SEQ ID NO:1; and the sgRNA has a nucleotide sequence shown in SEQ ID NO:2, or a modified sgRNA sequence based on SEQ ID NO: 2. The present invention can effectively solve the problems of heterologous codon bias and cytotoxicity in the application of SpCas9 in Lactobacillus paracasei. The gene editing is performed in cells or in vitro by introducing artificially designed CRISPR RNA and a repair template, which solves the problem of low electroporation efficiency caused by the strains' characteristics or relatively large carried plasmids, and has broad application prospects in the field of gene editing.

### TECHNICAL FIELD

The present invention relates to the field of gene editing, and in particular to a CRISPR/LpCas9 gene editing system and application thereof.

### BACKGROUND

CRISPR/cas9 system is an adaptive immune system found in bacteria and archaea and has been widely used as a gene editing tool.

In the CRISPR/cas9 system, Cas9 protein, crRNA, and tracrRNA are formed into a nucleic acid-protein complex for determining the binding of crRNA and a target sequence by recognizing a PAM sequence near a target site. When a functional PAM site is scanned, Cas9 is bound to a strand where non-PAM is located for examining the sequence matching between a spacer in the crRNA and a target DNA. If mismatching is not found in the seed region, then DNA double strands will be precisely cleaved by Cas9. CrRNA and tracrRNA are fused into a single RNA, which is called single guide (sgRNA). This fusion does not affect the efficiency of a system of crRNA and tracrRNA.

The application of a CRISPR system in lactic acid bacteria is still very limited. Currently, the CRISPR/Cas9 gene editing system is most widely used, which is constructed based on the SpCas9 of Streptococcus pyogenes as the core. The application of the CRISPR/Cas9 gene editing system in the lactobacillus is limited due to cytotoxicity, heterologous codon bias, etc.

Besides, a Cas9 protein expression vector is constitutively expressed. Plasmids used to construct a knockout system are too many or too large, which will affect the electroporation efficiency, and is also a factor that limits the application of the SpCas9 in the lactobacillus.

### SUMMARY

An object of the present invention is to solve at least the above problems, and to provide, at least, the advantages that will be described later.

Another object of the present invention is to provide a CRISPR/LpCas9 gene editing system, which is derived from a Cas9 gene editing tool of the lactic acid bacteria, that can effectively solve the problems of heterologous codon bias and cytotoxicity in the application of CRISPR gene editing in the lactic acid bacteria. In Lactobacillus paracasei, the problem of low electroporation efficiency caused by the strain's characteristics or relatively large carried plasmids can be solved by the introduction of artificially designed CRISPR RNA and a repair template.

To realize the objects mentioned above and other advantages, the present invention provides a CRISPR/LpCas9 gene editing system, and gene editing refers to gene editing in cells or in vitro. The CRISPR/LpCas9 gene editing system is a complex of LpCas9 protein and sgRNA, which can accurately locate a target DNA sequence and cleave DNA double strand, wherein, the LpCas9 protein has an amino acid sequence shown in SEQ ID NO:1; and the sgRNA has a nucleotide sequence shown in SEQ ID NO:2, or a modified sgRNA sequence based on SEQ ID NO: 2.

Preferably, the cells include eukaryotic cells and prokaryotic cells; wherein the eukaryotic cells include mammalian cells and plant cells; and the prokaryotic cells include Lactobacillus paracasei.

Preferably, the LpCas9 protein includes a LpCas9 protein variant with no cleavage activity, a LpCas9 protein variant with single-strand cleavage activity, and a LpCas9 protein variant with double-strand cleavage activity.

Preferably, the LpCas9 protein is obtained by codon optimization, transcription, and translation of a DNA sequence of an original LpCas9 protein, wherein detection cells are HEK293T cells, the original LpCas9 protein has a nucleotide sequence shown in SEQ ID NO: 3, and an optimized LpCas9 protein has a nucleotide sequence shown in SEQ ID NO: 4.

Preferably, the sgRNA is designed according to prediction results of crRNA and tracrRNA secondary structures.

Preferably, the accurate location of flanking of the target DNA sequence includes identifying a PAM sequence on the target DNA sequence by the complex of LpCas9 protein and sgRNA.

Preferably, the PAM sequence is TCAAAA, and the target DNA sequence is shown in SEQ ID NO: 5.

Preferably, the PAM sequence is TGTAAA, and the target DNA sequence is shown in SEQ ID NO: 5.

A kit of the CRISPR-LpCas9 gene editing system includes the LpCas9 protein, the sgRNA of the target DNA sequence, or the target DNA sequence.

A detection method of editing efficiency in gene editing of the CRISPR/LpCas9 gene editing system includes the following steps:

S1, designing a sgRNA of a target gene according to a PAM sequence of TCAAAA;

S2, cloning a LpCas9 gene sequence after human codon optimization and the corresponding sgRNA into an expression vector;

S3, transforming the expression vector into HEK293T cells;

S4, extracting a cell genome, design a primer, and performing PCR amplification of a DNA fragment in a gene editing site; and

S5, detecting the editing efficiency using the T7E1 enzyme.

The present invention includes at least the following substantial improvements and beneficial effects:

1. The present invention provides a kit of the CRISPR/LpCas9 gene editing system, and the kit includes the complex of LpCas9 protein and sgRNA.

2. The present invention provides the CRISPR/LpCas9 gene editing system, which can be effectively used in lactobacillus. Compared with the existing most widely used CRISPR/SpCas9 gene editing system from Streptococcus pyogenes, the CRISPR/LpCas9 gene editing system can effectively solve the problems of cytotoxicity and heterologous codon bias in the application of SpCas9 in the lactobacillus since LpCas9 is derived from Lactobacillus paracasei.

3. Only the sgRNA and a repair template need to be introduced by gene editing of an endogenous CRISPR system, which solves the problem of affecting electroporation efficiency caused by too many plasmids or too large plasmids and significantly improves the gene editing efficiency.

Other advantages, objects, and features of the present invention will be shown in part through the following description, and in part will be understood by those skilled in the art from study and practice of the present invention.

### BRIEF DESCRIPTION OF THE DRAWINGS

FIG. 1 is a figure of a CRISPR/LpCas9 gene editing system according to one technical solution of the present invention.

FIG. 2 is a figure showing rare codons analysis of an original LpCas9 protein sequence in HEK293T cells according to one technical solution of the present invention.

FIG. 3 is a figure showing rare codons analysis of an optimized LpCas9 protein sequence in HEK293T cells according to one technical solution of the present invention.

FIG. 4 is a figure of a dual fluorescent reporter system-pmT/mG system according to one technical solution of the present invention.

FIG. 5 is a figure of sgRNA according to one technical solution of the present invention.

FIG. 6 is a figure showing fluorescent detection results of the dual fluorescent reporter system-pmT/mG system according to one technical solution of the present invention.

FIG. 7 is a figure showing detection results of editing efficiency in gene editing of a LpCas9-mediated gene according to one technical solution of the present invention.

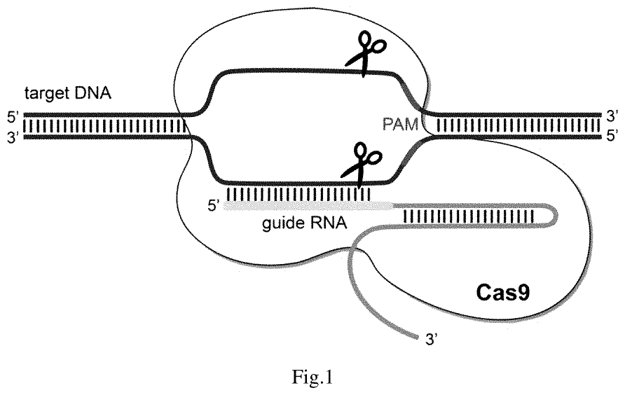

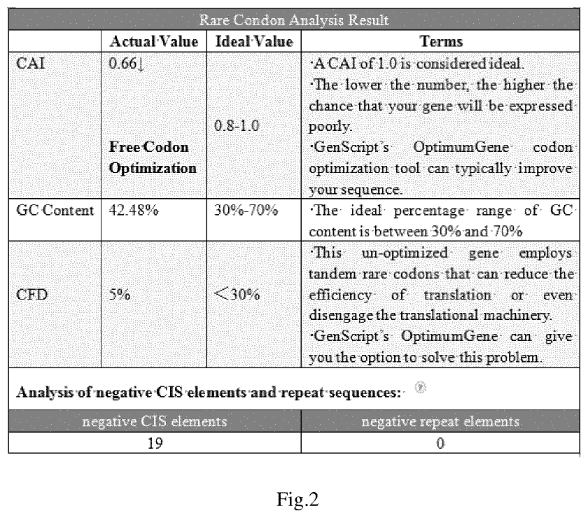

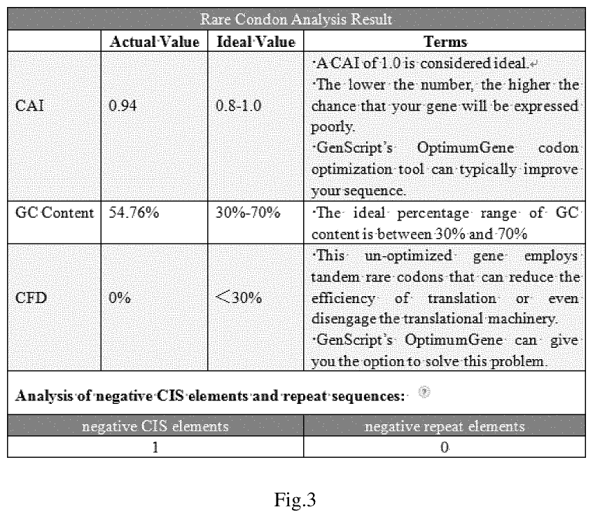

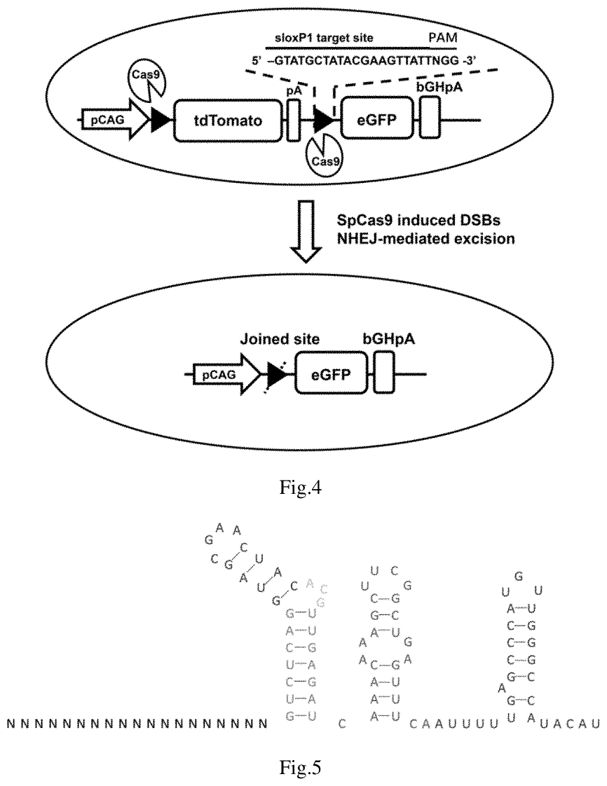

### DETAILED DESCRIPTION

The present invention will now be described in further detail with reference to the embodiments, in order to enable person skilled in the art to practice with reference to the literal description of the specification.

In the present invention, the CRISPR/LpCas9 gene editing system includes L. paracasei Cas9 (LpCas9) protein and sgRNA, and gene editing is realized by the combination of the L. paracasei Cas9 (LpCas9) protein and the sgRNA.

The L. paracasei Cas9 (LpCas9) protein belongs to a type-II system of Lactobacillus paracasei, and an amino acid sequence of the LpCas9 protein is shown in a sequence table (SEQ ID NO:1).

In the codon optimization of the LpCas9 protein, HEK293T cells are used as detection cells, a CAI (Codon Adaptation Index) of an original LpCas9 sequence in the HEK293T cells is 0.66, a normal range is 0.8-1.0, and 1.0 is considered as the most ideal value. The lower the CAI index, the more likely that the expression is poor. The GC content is 54.76%, and the ideal range of the GC content is 30% to 70%. If the GC content is beyond the ideal range, it will be considered to harm transcription and translation efficiency. A percentage of a low frequency (<30%) codon-based on a target host organism is 5%, an unoptimized sequence includes tandem rare codons, which may reduce the translation efficiency. Negative CIS elements usually refer to sequence motifs for negatively regulated gene expression at a transcription or translation level, and 19 Negative CIS elements have been found. Negative repeat elements usually refer to direct or inverted repeats that may affect gene synthesis or expression and have not been found. The CAI index of an optimized LpCas9 sequence in the HEK293T cells is 0.94, which significantly improves gene expression. The GC content of the genes is 54.76%. The percentage of the low frequency (<30%) codon-based on the target host organism is reduced to 0%, and the number of Negative CIS elements is reduced to 1. A codon of the original LpCas9 protein is shown in SEQ ID NO: 3, and a codon of the optimized LpCas9 protein is shown in SEQ ID NO: 4.

The sgRNA is formed as follows. In vivo, a locally paired structure is formed by tracrRNA and crRNA. The sgRNA sequence is formed by removing some non-functional parts in vitro and linking by a linker. Then gene synthesis is performed. A position of the tracrRNA is confirmed according to base-pairing by comparing four classic positions of tracrRNA (eight sequences in sense and antisense strands) with the repetitive sequences using MEGA7. A promoter is predicted using BPROM (http://www.softberry.com/berry.phtml?topic=bprom&group=programs&subgroup=gfindb), and a terminator is predicted using ARNold (http://rssf.i2bc.paris-saclay.fr/toolbox/arnold/index.php).

The sgRNA sequence is: NNNNNNNNNNNNNNNNNNNNGUCUCAGGUAGCGAACUACACGUUGA GAUCAAACAAAGCUUCGGCUGAGUUUCAAUUUUUGAGCCCAUGUUG GGCCAUACAU (SEQ ID NO 2); wherein the consecutive N in the sgRNA sequence refers to a sequence that can be designed according to a target gene in the application.

A PAM sequence is TCAAAA or TGTAAA, the target DNA sequence is GAATAACTTCGTATAGCATAC (SEQ ID NO 5).

In one embodiment, the CRISPR/LpCas9 gene editing system of the present invention is detected using a dual fluorescent reporter system-pmT/mG system, and the present invention adopts two vectors of pmT/mG-PAMn vector and pX330-sg-lp vector.

Detection principle: the dual fluorescent reporter system-pmT/mG system adopts a tandem dimer tomato fluorescent protein (tdTomato) and an enhanced green fluorescent protein (eGFP) as reporter gene proteins, wherein a blank triangle refers to a sloxP sequence locating at both sides of the tdTomato. Both sides of the two sloxP sequences can be replaced with different PAM sequences according to requirements. When a functional PAM sequence is recognized, a sloxP region will be cleaved by the Cas9 protein to break two double strands, then a non-homologous end joining will occur, two sites will be linked, and a CAG promoter will directly start the expression of the eGFP such that green fluorescent cells can be detected under a fluorescence microscope.

### Specific Detection Embodiments

1. Preparation of Medium and Solution

(1) Preparation of LB Medium:

Adding 10 g tryptone, 5 g yeast extract, and 10 g NaCl to 950 mL deionized water; stirring on a magnetic stirrer for complete dissolution; adjusting pH to 7.0 with a 5 mol/L NaOH solution; diluting with deionized water to 1 L (wherein a solid medium needs to be added with 1.5%-2% agar powder, then be diluted to a constant volume); and performing steam sterilization under 121° C. for 20 min.

(2) Preparation of ddH₂O:

Ultrapure water is subjected to steam sterilization under 121° C. for 20 minutes, then filtered with a disposable 0.22 μm filter membrane and packed, it can be stored in a refrigerator at −20° C. for several months.

(3) Preparation of an Ampicillin Solution

The Ampicillin solution has a concentration of 100 mg/mL. Take 5 g Ampicillin, put it in a 50 mL centrifuge tube, add 40 mL of sterile water, mix, dissolve, then dilute to 50 mL, filter it with a disposable 0.22 μm filter membrane to sterilize, pack it into small aliquots (about 0.5 mL/tube), and store it in a refrigerator at −20° C. for several months. Repeated freezing and thawing should be avoided.

(4) Preparation of DMEM High Glucose Complete Medium Containing 10% Serum:

Adding 10 mL of fetal bovine serum and 1 mL of a Penicillin-Streptomycin solution into 89 mL of DMEM high glucose complete medium without serum, and storing at 4° C. after sealing with parafilm.

2. Replacement and Insertion of SgRNA

(1) A SgRNA fragment of Streptococcus pyogenes in pX330-sloxP3 is replaced with a designed L. paracasei sgRNA (Lp sgRNA) using a SnapGene software, and a sequence from a PciI restriction enzyme cutting site to an XbaI restriction enzyme cutting site is exported to perform gene synthesis by Beijing Genomics Institute.

(2) Transformation and Amplification of the Vector

A cloning vector (pMV-lp sgRNA) of synthetic sgRNA fragment and pX330-sloxP3 are transformed into DH5α competent cells of Escherichia coli for amplification, respectively. The specific operation process includes the following steps:

(a) placing 50 μL of competent cells in a centrifuge tube into an ice bath, adding less than 0.5 ng of a target DNA to the competent cell suspension (adding 5 μL at most calculated based on plasmid concentration), mixing gently, and standing in the ice bath for 30 min;

(b) placing the centrifuge tube in a 42° C. water bath to heat shock for 60-90 sec, and then quickly transfer it to the ice for cooling for 2-3 min;

(c) adding 950 μL of a sterile antibiotic-free LB liquid medium to the centrifuge tube, mixing, placing it on a 37° C. shaker, and performing shake culture at 150 rpm for 45 minutes to recover the competent cells;

(d) mixing the competent cell suspension in the centrifuge tube, taking 100 μL onto an LB solid agar medium containing corresponding antibiotics, and spreading gently and evenly with a sterile spreading rod, placing the plate at room temperature until the competent cells suspension is absorbed, and performing inverted cultivation at 37° C. for 12-16 h;

(e) picking a single colony into 5-10 mL of an LB liquid medium using a sterilized white pipette tip, culturing under 200 rpm at 37° C. for 6-8 h for amplification.

(3) Rapid Extraction of Plasmids

The competent cells suspension after amplification by step (2) is subjected to plasmid extraction at room temperature using a rapid plasmid extraction kit, including the following steps:

(a) centrifuging 5 mL of the competent cells suspension at 12,000 rpm for 1 min several times, and collecting precipitate into a 2 mL centrifuge tube;

(b) adding 150 μL of a solution P1 (RNase A and TIANRed are added before use, and the color of the solution is red) into the centrifuge tube, and suspending thoroughly the precipitate using a pipette and shaker;

(c) adding 150 μL of a solution P2 into the centrifuge tube, flipping up and down gently for 6-8 times to fully lyse the competent cells (the color of the solution in the centrifuge tube is turned into clear purple);

(d) adding 350 μL of a solution P5 into the centrifuge tube, immediately and quickly mixing 10-20 times until flocculent precipitation appears, and centrifuging at 12,000 rpm for 2 min to obtain clear yellow supernatant;

(e) transferring the supernatant to an adsorption column CP3 (the adsorption column is placed in a collection tube) using a pipette (be careful not to suck out the precipitation), centrifuging at 12,000 rpm (˜13,400×g) for 30 sec, discarding waste liquid in the collection tube, and putting the adsorption column CP3 in the collection tube;

(f) adding 300 μL of a washing solution PWT (added absolute ethanol) to the adsorption column CP3, centrifuging at 12,000 rpm (˜13,400×g) for 30 sec, discarding waste liquid in the collection tube, and putting the adsorption column CP3 in the collection tube;

(g) centrifuging at 12,000 rpm (˜13,400×g) for 1 min for removing residual washing solution PWT in the adsorption column CP3;

(h) placing the adsorption column CP3 in a clean centrifuge tube, dropping 50 μL of ddH₂O preheated at 60° C. to a central part of an adsorption membrane, centrifuging at 12,000 rpm (˜13,400×g) for 30 sec, and collecting a plasmid solution into the centrifuge tube;

(i) measuring a concentration of the plasmid solution using a microplate reader, and then storing it in a −20° C. refrigerator.

(4) Preparation of Enzyme Fragments

Both pMV-lpsgRNA and pX330-sloxp3 are subjected to double enzyme digestion with PciI and XbaI, and react in a PCR instrument at 37° C. for 30 minutes. An enzyme digestion system is shown in Table 1:

TABLE 1

| Components | Usage |
|---|---|
| Buffer 3.1 | 5 μL |
| PciI | 1 μL |
| XbaI | 1 μL |
| Vector | Less than 1 ng |
| ddH₂O | up to 50 μL |

The obtained enzyme fragments are subjected to band separation by agarose gel electrophoresis; and for the separated enzyme fragments, short fragments of pMV-lpsgRNA vector (synthetic fragments) and long fragments of pX330 vector (backbone parts) are recycled at room temperature using a DNA purification and recovery kit.

The above two recycled fragments are linked and react in the PCR instrument at 16° C. for 30 minutes-2 h. The linking system is shown in Table 2:

TABLE 2

| Components | Usage |
|---|---|
| T4 Buffer | 1 μL |
| T4 ligase | 1 μL |
| Fragments | 4 μL |
| Vector | 2 μL |
| ddH₂O | up to 10 μL |

The obtained linked product after the above linking is transformed into DH5α competent cells, and a constructed vector is named as pX330-sg.

Sequencing verification is performed using a pX330-sg-F primer.

pX330-sg-F:
(SEQ ID NO 6)
GGGAAACGCCTGGTATCTTT

3. Replacement of LpCas9

The initiation codon and termination codon at both ends of an optimized Cas9 sequence are removed, the Cas9 protein sequence of Streptococcus pyogenes of pX330-sg is replaced using SnapGene software, and a sequence of a LpCas9 position from AgeI restriction enzyme cutting site to the EcoRI restriction enzyme cutting site is exported to perform gene synthesis by Beijing Genomics Institute (BGI).

A vector (pMV-LpCas9) of synthetic sequence and pX330-sg are transformed into the DH5α competent cells for amplification, respectively. The specific operation processes are the same as those in replacement and insertion of sgRNA.

Both pMV-LpCas9 and pX330-sg are subjected to double enzyme digestion with AgeI-HF and EcoRI-HF, respectively, and react in the PCR instrument at 37° C. for 30 minutes. An enzyme digestion system is shown in Table 3:

TABLE 3

| Components | Usage |
|---|---|
| Buffer Cut Smart (10X) | 5 μL |
| AgeI-HF | 1 μL |
| EcoRI-HF | 1 μL |
| Vector | Less than 1 ng |
| ddH₂O | up to 50 μL |

The specific separation processes of DNA fragments are the same as those of replacement and insertion of sgRNA, and 4263 bp fragments of the pMV-LpCas vector (LpCas9 synthetic fragments) and 4209 bp fragments of pX330 vector (backbone parts) are recycled according to corresponding steps in the replacement and insertion of sgRNA. The 4263 bp fragments and 4209 bp fragments after recycling are linked and react in the PCR instrument at 16° C. for 30 minutes, wherein the linking system is the same as those of the replacement and insertion of sgRNA.

The linked product is transformed into the DH5α competent cells, and a constructed fragment is named as pX330-sg-lp.

Sequencing verification is performed using a pX330-cas9-F1 primer, a pX330-cas9-F2 primer, a pX330-cas9-F3 primer, pX330-cas9-R1 primer, pX330-cas9-R2 primer, and pX330-cas9-R3 primer.

pX330-cas9-F1:
(SEQ ID NO 7)
CGCTCCGAAAGTTTCCTT
pX330-cas9-F2:
(SEQ ID NO 8)
TGCTGAAGCAGAGAAACAAG
pX330-cas9-R1:
(SEQ ID NO 10)
GGTAGGGCATGCTCTTTCT
pX330-cas9-F3:
(SEQ ID NO 9)
TGGAGACAGTGGCATGA
pX330-cas9-R2:
(SEQ ID NO 11)
AATCTGCTCGGTCAGCT
pX330-cas9-R3:
(SEQ ID NO 12)
CAAACAACAGATGGCTGGCA.

4. Series Vectors Construction of pmT/mG-PAMn

A pmTmG vector sequence is complicated, and the AgeI restriction enzyme cutting site is introduced by a method that a sequence between BglII and MfeI is subjected to gene synthesis by BGI.

Series vectors construction of pmT/mG-PAMn includes the following steps:

(1) Synthesis of a Short Fragment

A short fragment containing two consecutive sloxp3-PAM sequences is synthesized. The two consecutive sloxp3-PAM sequences are located between the EcoRI-MluI restriction enzyme cutting site and AgeI-BglII restriction enzyme cutting site, respectively. A sequence between the MluI restriction enzyme cutting site and AgeI restriction enzyme cutting site is used to connect two fragments.

(2) pmT/mG-NT-PAM

Both pMV-PAM vector and pmTmG-M vector are subjected to double enzyme digestion with BglII and EcoRI and react in the PCR instrument at 37° C. for 30 minutes. An enzyme digestion system is shown in Table 4:

TABLE 4

| Components | Usage |
|---|---|
| Buffer 3.1 | 5 μL |
| BglII | 1 μL |
| EcoRI | 1 μL |
| Vector | Less than 1 ng |
| ddH₂O | up to 50 μL |

DNA bands of the above enzyme digestion system are separated. 296 bp fragment (PAM synthetic fragment) of pMV-PAM vector and 7442 bp fragment (backbone parts) of pmTmg-M vector are recycled, and the recycled 296 bp fragment and 7442 bp fragment are linked. A constructed vector is named pmTmG-NT-PAM.

(3) Screening of Positive Clones by Bacterial Colony PCR and Sequencing Verification

pmTmG-NT-PAM is subjected to a screening of positive clones using a bacterial colony (pTG-2991F: GATGAACTTCAGGGTCAGCTT (SEQ ID NO 13); pTG-2988R: GGGACTTCCTTTGTCCCAAATC (SEQ ID NO 14)) PCR. A reaction system is shown in Table 5

TABLE 5

| Components | Usage | |
|---|---|---|
| 2 × Taq PCR MasterMix | 12.5 | μL |
| pTG-2991F | 1 | μL |
| pTG-2988R | 1 | μL |

| | |
|---|---|
| ddH₂O | up to 25 μL |

The reaction procedure is as follows:

| | | | |
|---|---|---|---|
| 94° C. | 10 min | —— | 1 cycle |
| 94° C. | 30 s | | 25-30 cycle |
| | | } | |
| 60° C. | 30 s | | |
| 72° C. | 1 min/kb | | |
| 72° C. | 10 min | | 1 cycle |
| 4° C. | | | 1 cycle |

(2) Sequencing Verification is Performed Using a pTG-2991F Primer.

pmTmG-PAM

(1) Both pmTmG-M vector and pmTmG-NT-PAM vector are subjected to double enzyme digestion with AgeI-HF and MluI-HF, and an enzyme digestion system is shown in Table 6:

TABLE 6

| Components | Usage |
|---|---|
| Buffer CutSmart (10X) | 5 μL |
| AgeI-HF | 1 μL |
| MluI-HF | 1 μL |
| Vector | Less than 1 ng |
| ddH₂O | up to 50 μL |

(2) DNA bands of the above enzyme digestion system are separated. 2355 bp fragment of the pmTmG-M vector (containing tdTOMATO fragment) and 7523 bp fragment of pmTmG-NT-PAM vector (backbone parts, the PAM sequence has been replaced) are recycled. The recycled 2355 bp fragment and 7523 bp fragment are linked, and a constructed vector is named pmTmG-PAM.

(3) pmTmG-NT-PAM is subjected to screening of positive clones using a bacterial colony PCR, and sequencing verification is performed using a pTG-2991F primer.

Cultivation and transfection of HEK293T cells, wherein plasmids used for transfection are treated with endotoxin removal.

1. Cell culture and passage

(1) taking 1 bottle of HEK293T cells, and discarding medium;

(2) adding 2 mL PBS into each bottle, washing 2 times, and removing residual serum;

(3) adding 1 mL of 0.25% EDTA-trypsin into each bottle, gently shaking to soak all cells by trypsin, and digesting at 37° C. for 3-4 min;

(4) adding 2 mL of a cell medium containing serum, gently blowing the bottle wall to detach the cells;

(5) transferring to a 15 mL centrifuge tube, centrifuged at 1500 rpm for 4 minutes, discarding supernatant;

(6) adding 4 mL of the cell medium containing serum to the centrifuge tube, blowing and mixing;

(7) dividing evenly the cell suspension into 3 cell culture flasks, and adding the cell medium into each cell culture flask until the cell medium reaches 5 mL, mixing and placing it in a 37° C. CO₂ incubator for culture.

2. Cell Transfection

(1) performing cell plating for the 293T cells at the day before an experiment, observing a density of the 293T cells under a microscope the next day, and performing cell transfection when the density reaches 70%-80%;

(2) adding 500 ng plasmids to an EP tube, then adding 200 μL of a serum-free DMEM medium, shaking and mixing;

(3) adding 2 μL of a Tubofect transfection reagent to the EP tube, and mixing immediately to obtain a mixed liquid;

(4) adding the mixed liquid into the pore plate for cell culture, and culturing in the incubator;

(5) replacing medium after 6 h;

(6) observing transfection results using a fluorescence microscope after 24 h.

PAM Activity Verification Results

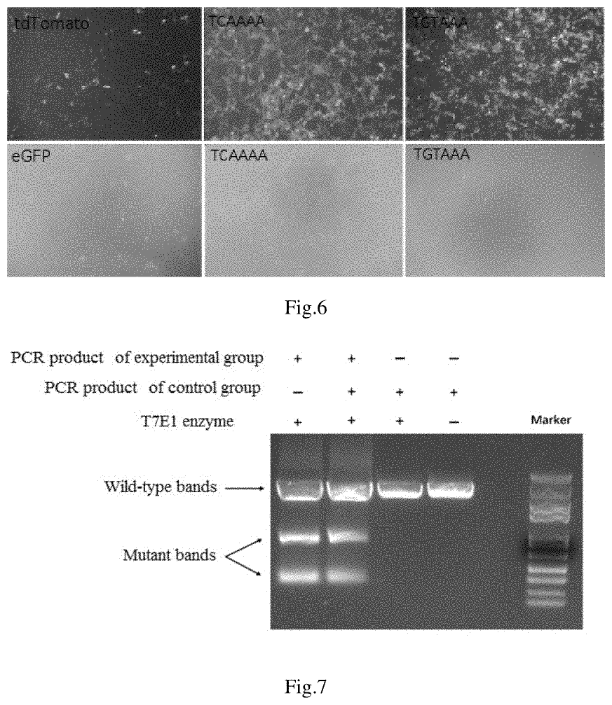

The pmT/mG-PAMn vector containing two PAM sequences of TCAAAA and TGTAAA, and pX330-sg-lp vector is co-transfected into the 293T cells, and the statistical results of fluorescent cells are shown in Table 7 and FIG. 6. It can be seen that eGFP is positive, which shows that the functional PAM is recognized by LpCas9. That is, gene editing of the target DNA is achieved.

TABLE 7

| | | Red fluorescent | Green fluorescent |
|---|---|---|---|
| No. | Groups | cells | cells |
| 1 | TCAAAA | Yes | Yes |
| 2 | TGTAAA | Yes | Yes |
| Control 1 | pmTmG | Yes | No |
| | control | | |
| Control 2 | peGFP | No | Yes |
| | control | | |
| Control 3 | Blank | No | No |
| | control | | |

Detection of the Editing Efficiency in Gene Editing of the CRISPR/LpCas9 Gene Editing System:

Detection method: LpCas9 is used to edit the HEK293T cell genomes and T7E1 (T7 endonuclease 1) is used to detect the editing efficiency.

The method includes the following specific steps:

S1, designing a sgRNA sequence (20 nt) for the HEK293T cell genome target locus EMX1 using two web tools of CHOPCHOP (https://chopchop.cbu.uib.no/) and Benchling (https://www.benchling.com), wherein the PAM sequence recognized by LpCas9 is TCAAAA, and designed sgRNA sequence is sgRNA1 (5′-gtatcctgcttcattaacta-3′);

S2, cloning the designed sgRNA into a LpCas9 expression vector;

S3, transfecting the LpCas9 expression vector into HEK293T cells (no repair template, cells are repaired with NHEJ) using TurboFect™ Transfection Reagent, wherein the transfection operation is carried out according to supplier's instructions, and obtained cells after transfecting the expression vector into cells for 72 h are used for the editing efficiency analysis;

S4, extracting a cell genome, design a primer, and performing PCR amplification of a DNA fragment in a gene editing site; and

wherein the primer is designed as follows:

T7E1 F
(SEQ ID NO 15)
(5′-CTCCCTTTTCCCTCCTGGCA-3′)
T7E1 R
(SEQ ID NO 16)
(5′-AGCCAGTTTTGGGGAGGCAG-3′)

wherein a distance between T7E1 F and a gene editing site is 379 bp, and a distance between T7E1 R and the gene editing site is 729 bp.

For the specific system and conditions of the PCR amplification please refer to the actual PCR polymerase instructions. The PCR amplification requires a high-fidelity polymerase, and Q5 high-fidelity polymerase is used for PCR amplification in the experiment.

PCR products of an experimental group use a cell genome of an expression vector for transfection as a PCR template; the PCR products of a control group use a cell genome of an empty vector for transfection as a PCR template.

The preparation of an annealing reaction system is shown in Table 8;

TABLE 8

| Tube number | 1 | 2 | 3 | 4 |
|---|---|---|---|---|
| PCR products (μL) of | 18 | 9 | 0 | 0 |
| experimental group | | | | |
| PCR products (μL) of | 0 | 9 | 18 | 18 |
| control group | | | | |
| 10X NEB 2.1 Buffer (μL) | 2 | 2 | 2 | 2 |
| Total (μL) | 20 | 20 | 20 | 20 |

Perform heat denaturation and annealing renaturation treatment;

Perform denaturation and annealing treatment using a PCR instrument, a setting procedure is as follows:

95° C. 5 min

94° C. 2 sec, −0.1° C./cycle, 200 times

75° C. 1 sec, −0.1° C./cycle, 600 times

16° C. 2 min

S5, T7E1 enzyme digestion reaction.

T7E1 is an endonuclease that can recognize a mismatch position and cut the mismatch position to produce two or more DNA fragments, and the DNA fragments can be separated by gel electrophoresis. To facilitate gel separation and subsequent analysis, when a sequence to be detected is subjected to PCR amplification, a distance difference between an expected editing site and two sides of PCR products is no less than 100 bp. That is, as described in the S4, the distance difference between the expected editing site and two sides of PCR products is no less than 300 bp.

The reaction systems in the No. 1, 2, and 3 tubes are added with 2 μL of T7E1 enzyme (5 u/μL), and react at 37° C. for 45 min. All tubes are added with 3-4 μL 6× Gel Loading Dye, Purple (6×, NEB), to terminate the reaction. 20 μL of reaction products are immediately subjected to 2% TAE agarose gel electrophoresis after terminating the reaction, and a Marker used is 100 bpplus II DNA Ladder (from TransGen Biotech).

After a gel electrophoresis map is obtained, gray values of a wild-type bands and mutant bands are measured using ImageJ gel quantification software for detecting the gene editing efficiency of the LpCas9-mediated gene, wherein the gray value of the wild-type bands is a, and gray values of the mutant bands are b and c.

The ratio of an enzyme digestion fragment is: f_{cut}=(b+c)/(a+b+c).

The following formula can be used to estimate the editing efficiency based on a binomial probability distribution formed by a double-stranded structure:

Indel (%)=100×(1−\sqrt{(1−f_{cut})})

The result is shown as FIG. 7, wherein, the wild-type band is about 1100 bp, the mutant bands are about 379 bp and 729 bp, respectively and the editing efficiency is 20.2915%.

Although the embodiments of the present invention have been disclosed above, they are not limited to the applications previously mentioned in the specification and embodiments and can be applied in various fields suitable for the present invention. For an ordinary skilled person in the field, other changes may be easily achieved. Therefore, without departing the general concept defined by the claims and their equivalents, the present invention is not limited to particular details and embodiments shown and described herein.

What is claimed is:

1. A CRISPR/LpCas9 gene editing system, the gene editing referring to gene editing in cells or in vitro, being characterized in that, the CRISPR/LpCas9 gene editing system is a complex of LpCas9 protein and sgRNA, which can accurately locate a target DNA sequence and cleave DNA double strands, wherein, the LpCas9 protein has an amino acid sequence shown in SEQ ID NO:1; and the sgRNA has a nucleotide sequence shown in SEQ ID NO:2, or a modified sgRNA sequence based on SEQ ID NO: 2.

2. The CRISPR/LpCas9 gene editing system according to claim 1, being characterized in that, the cells comprise eukaryotic cells and prokaryotic cells; wherein the eukaryotic cells comprise mammalian cells and plant cells, and the prokaryotic cells comprise Lactobacillus paracasei.

3. The CRISPR/LpCas9 gene editing system according to claim 1, being characterized in that, the LpCas9 protein comprises a LpCas9 protein variant with no cleavage activity, a LpCas9 protein variant with single-strand cleavage activity and a LpCas9 protein variant with double-strand cleavage activity.

4. The CRISPR/LpCas9 gene editing system according to claim 1, being characterized in that, the LpCas9 protein is obtained by codon optimization, transcription, and translation of a DNA sequence of an original LpCas9 protein, wherein detection cells are HEK293T cells, the original LpCas9 protein has a nucleotide sequence shown in SEQ ID NO: 3, and an optimized LpCas9 protein has a nucleotide sequence shown in SEQ ID NO: 4.

5. The CRISPR/LpCas9 gene editing system according to claim 1, being characterized in that, the sgRNA is designed according to prediction results of crRNA and tracrRNA secondary structures.

6. The CRISPR/LpCas9 gene editing system according to claim 1, being characterized in that, accurate location of flanking of the target DNA sequence comprises identifying a PAM sequence on the target DNA sequence by the complex of LpCas9 protein and sgRNA.

7. The CRISPR/LpCas9 gene editing system according to claim 6, being characterized in that, the PAM sequence is TCAAAA, and the target DNA sequence is shown in SEQ ID NO: 5.

8. The CRISPR/LpCas9 gene editing system according to claim 6, being characterized in that, the PAM sequence is TGTAAA, and the target DNA sequence is shown in SEQ ID NO: 5.

9. A kit of the CRISPR-LpCas9 gene editing system according to claim 1, comprises the LpCas9 protein, the sgRNA of the target DNA sequence, or the target DNA.

10. A detection method of editing efficiency in gene editing of the CRISPR-LpCas9 gene editing system according to claim 1, being characterized in that, the detection method comprises the following steps:
S1, designing a sgRNA of a target gene according to a PAM sequence of TCAAAA;
S2, cloning a LpCas9 gene sequence after human codon optimization and the corresponding sgRNA into an expression vector;
S3, transforming the expression vector into HEK293T cells;
S4, extracting a cell genome, design a primer, and performing PCR amplification of a DNA fragment in a gene editing site; and
S5, detecting the editing efficiency using a T7E1 enzyme.
